(19) United States
(12) Patent Application Publication
(10) Pub. No.: US 2013/0204236 A1
(43) Pub. Date: Aug. 8, 2013

(54) METHOD AND SYSTEM FOR LASER OCULAR SURGERY

(71) Applicant: Nanophthalmos, LLC, Miami, FL (US)

(72) Inventor: Richard Awdeh, Miami Beach, FL (US)

(73) Assignee: Nanophthalmos, LLC, Miami, FL (US)

(21) Appl. No.: 13/692,965

(22) Filed: Dec. 3, 2012

Related U.S. Application Data
(60) Provisional application No. 61/565,953, filed on Dec. 1, 2011.

(51) Int. Cl. A61F 9/008

(52) U.S. Cl. CPC A61F 9/00802
USPC 606/4

(57) ABSTRACT

A method of laser ocular surgery for treating glaucoma is disclosed. The method can include imaging a treatment eye to obtain an image of the treatment eye and aligning a laser on a region of the treatment eye based on the image of the treatment eye. The method can also include firing a plurality of laser pulses from the laser to ablate tissue in the region of the treatment site, wherein the tissue ablation creates micro-perforations in the region of the treatment site to incite an inflammatory reaction.

### CROSS-REFERENCE TO RELATED APPLICATIONS

This application claims the benefits of priority under 35 U.S.C. §§119 and 120 to U.S. Provisional Application No. 61/565,953, filed on Dec. 1, 2011, the entirety of which is incorporated herein by reference.

### TECHNICAL FIELD

The present disclosure is directed towards laser ocular surgery, and more particularly, the use of a laser ocular surgery for treating glaucoma.

### BACKGROUND

Glaucoma is caused by the body's inability to drain the clear, transparent liquid called the aqueous humor. Aqueous humor flows through the inner eye continuously. Typically, the aqueous fluid drains from the anterior chamber to the sclera, through a variety of drainage channels or canals. However, these channels can become smaller with age or become clogged by deposits. Inadequate drainage of the aqueous humor from the anterior chamber can lead to an abnormally high fluid pressure results within the eye. This is referred to as glaucoma. The high fluid pressure can lead to a slow loss of peripheral vision and eventually blindness.

Traditional glaucoma treatments can include forming a channel in the sclera of the eye to drain aqueous fluid from the anterior chamber of the eye, reducing fluid pressure. Typically, the channel in the sclera is made by a knife or other mechanical devices. These mechanical devices can cause trauma to the scleral tissue, resulting in scar tissue formation that can eventually obstruct the channel.

It is accordingly an object of the present disclosure to provide an improved system and method for reducing the high fluid pressure in the anterior chamber of the eye.

### SUMMARY

In accordance with the present disclosure, one aspect of the present disclosure is directed to a method of laser ocular surgery for treating glaucoma. The method can include imaging a treatment eye to obtain an image of the treatment eye and aligning a laser on a region of the treatment eye based on the image of the treatment eye. The method can also include firing a plurality of laser pulses from the laser to ablate tissue in the region of the treatment site, wherein the tissue ablation creates micro-perforations in the region of the treatment site to incite an inflammatory reaction.

In another embodiment, a system can be configured for laser ocular surgery. The system can include an imaging device configured to image a portion of a treatment eye, and a laser configured to generate a beam having power sufficient to create micro-perforations in the portion of the treatment eye. The system can also include an interface device coupling the laser to the treatment eye, wherein the interface device is configured to adjust a path of the beam based on output from the imaging device

Additional objects and advantages of the present disclosure will be set forth in part in the description which follows, and in part will be obvious from the description, or may be learned by practice of the present disclosure. The objects and advantages of the present disclosure will be realized and attained by means of the elements and combinations particularly pointed out in the appended claims.

It is to be understood that both the foregoing general description and the following detailed description are exemplary and explanatory only and are not restrictive of the present disclosure, as claimed.

The accompanying drawings, which are incorporated in and constitute a part of this specification, illustrate several embodiments of the present disclosure and together with the description, serve to explain the principles of the present disclosure.

### BRIEF DESCRIPTION OF THE DRAWINGS

FIG. 1 is schematic diagram of an ocular surgical system, according to an exemplary embodiment.

FIG. 2 is a flow diagram illustrating a method of ocular surgery, according to an exemplary embodiment.

FIG. 3 is a flow diagram illustrating a method of ocular surgery, according to another exemplary embodiment.

FIG. 4 is a diagram of a part of an eye.

Reference will now be made in detail to the present embodiments of the present disclosure, an examples of which are illustrated in the accompanying drawings. Wherever possible, the same reference numbers will be used throughout the drawings to refer to the same or like parts.

### DETAILED DESCRIPTION

It is understood that the embodiments described herein are not limited thereto. Those having ordinary skill in the art and access to the teachings provided herein will recognize additional modifications, applications, embodiments, and substitution of equivalents that all fall with the scope of the present disclosure. Accordingly, the present disclosure is not limited by the foregoing or following descriptions.

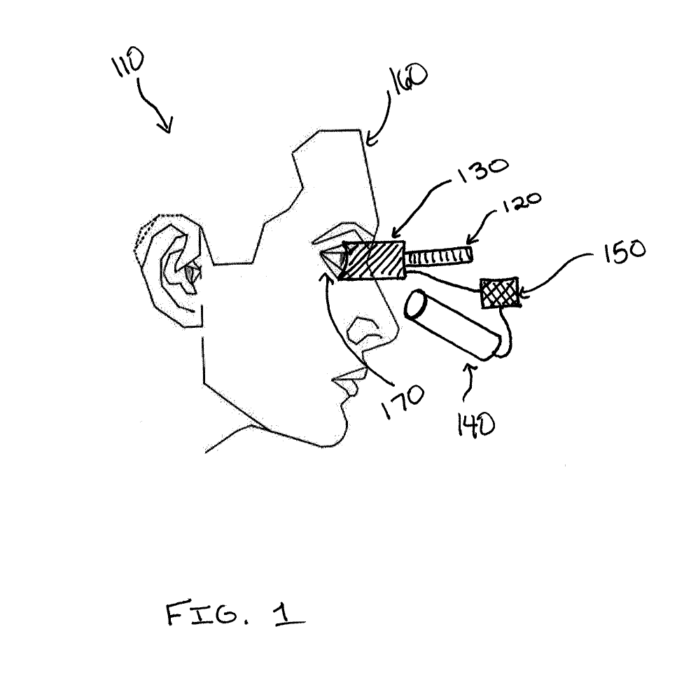

FIG. 1 is a schematic diagram of a surgical system 110 for performing ocular surgery using a laser, according to an exemplary embodiment. Surgical system 110 can comprise a laser 120, an interface device 130, an imaging device 140, and a controller 150. Laser 120 can include a femtosecond laser. In some aspects, laser 120 can include a type of laser configured to create micro-perforations in a portion of a treatment eye. For example, laser 120 can have sufficient power to form a plurality of micro-perforations in a trabecular meshwork of a treatment eye. Laser 120 can be further configured to ablate part of the treatment eye. Such treatment can be used to provoke an immune response to aid remodeling of tissue associated with the treatment eye.

Interface device 130 can be configured to couple patient 160 to laser 120. Device 130 can include attachments (not shown) configured to contact patient 160 to ensure laser 120 remains secure during a procedure. When coupled, interface device 130 can be positioned between laser 120 and a treatment eye 170 of patient 160.

In some embodiments, interface device 130 can comprise a mirror, a reflective substrate, or equivalent substrate configured to optically direct the path of laser 120 towards the treatment site of treatment eye 170. The treatment site can include the anterior segment of the eye, the trabecular meshwork, or the anterior sclera. Other regions of the eye may also be treated.

Imaging device 140 can comprise an optical coherence tomography (OCT) device, Schiemflug imaging device, or other equivalent imaging device capable of capturing images of ocular anatomy. Various other imaging devices may also be used. In addition, controller 150 can be part of imaging device 140 or can be a separate device. Controller 150 can be configured to receive data from imaging device 140 and output a signal to orient the mirror or reflective substrate of interface device 130 to direct laser 120 at the treatment site.

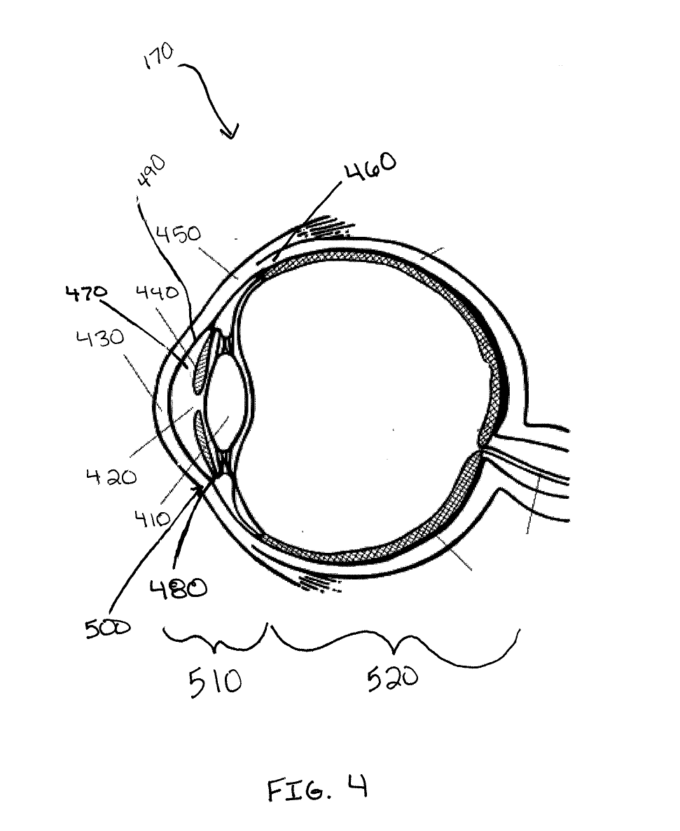

FIG. 4 shows a diagram of an eye 170, which is used to describe the method according to an exemplary embodiment. The eye comprises a lens 410, a pupil 420, a cornea 430, an iris 440, a conjunctiva 450, a sclera (anterior) 460, an anterior chamber 470, a travecular meshwork 480, a Schwalbe's line 490, a corneal limbus 500, an anterior segment 510, and a posterior segment 520.

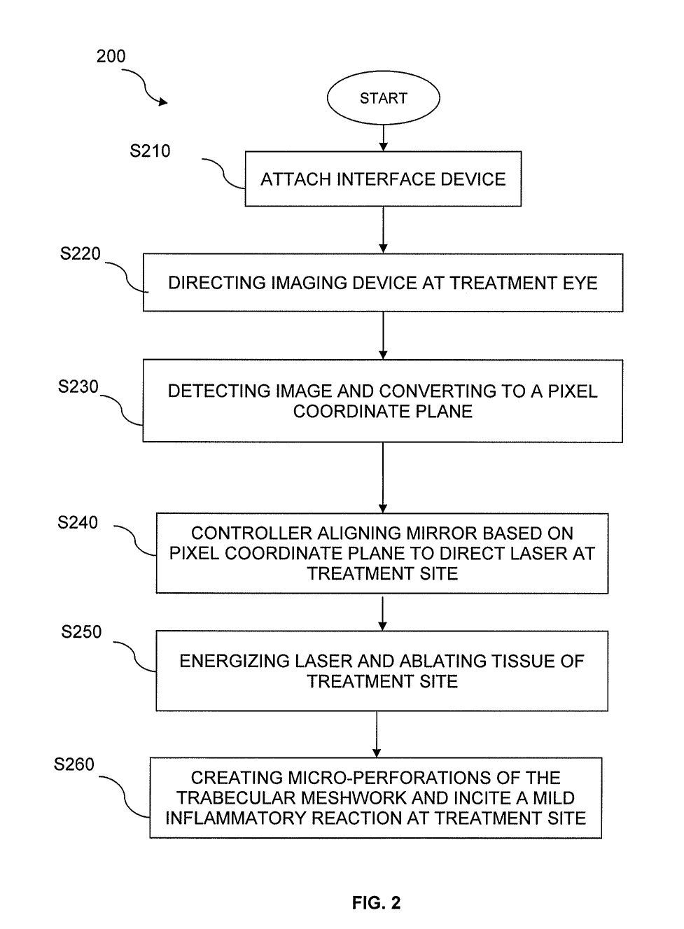

FIG. 2 shows a flow chart 200, for a method of performing ocular surgery, according to an exemplary embodiment. The first step, S210, can comprise attaching interface device 130 as described in relation to FIG. 1. Attaching interface device 130 can comprise aligning the interface device between laser 120 and treatment eye 170. Interface device 130 can be configured to expose anterior segment 510 of treatment eye 170 when attached. After completing step S210, the next step S220, can comprises directing imaging device 140 at the treatment eye 170.

Once Step S220 is completed, step S230 can comprise using imaging device 140 to detect and register the image and convert the image to a pixel coordinate plane by way of the imaging device software program. Following step S230, step S240 can comprise the controller 150 using the pixel coordinate data to align the mirror or reflective substrate of interface device 140 towards the treatment site. Controller 150 can be integrated into imaging device 140 and can include processor, computer readable data, and software programming. The treatment site can include the trabecular meshwork and angle of anterior segment 510 of treatment eye 170.

Next, step S250, can comprise laser 120 being energized and aimed at this tissue plane of the treatment site and the tissue can be ablated with various spot sizes of laser 120, in a 180 or 360 degree fashion. In other example, arcs of less than 360 degrees may be created on or about a region of the treatment eye.

Following step S250, step S260 can comprise creating micro-perforations of the travecular meshwork 480. These can be used to incite a mild inflammatory reaction in order to recruit macrophages and trabecular meshwork cells to the treatment site in order to initiate tissue remodeling at the treatment site.

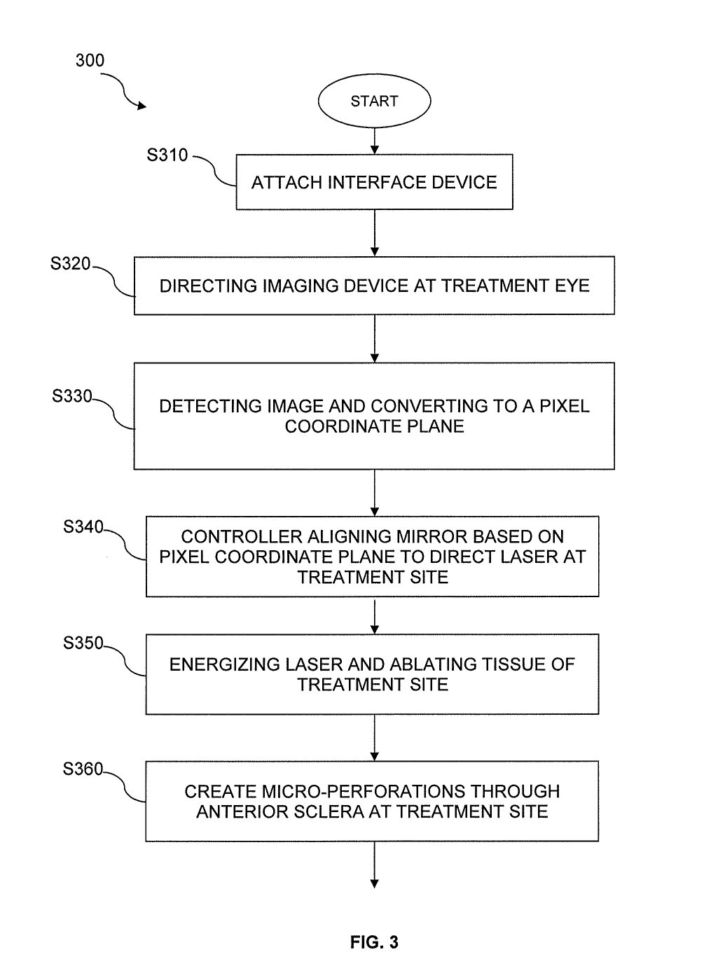

In another embodiment, a method similar to that described above in relation to FIG. 2 is shown in FIG. 3 as flow chart 300. The method described in flow chart 300 uses an interface device 130 configured to extend beyond the corneal limbus 500 to allow laser 120 to target the anterior sclera 460, adjacent to the Schwalbe's line 490 and the trabecular meshwork 480 of treatment eye 170. In alternative embodiments (not shown), a different device than interface device 130 can be used to target laser 120 to the treatment site.

The method of flow chart 300 can begin with Step S310, which comprises attaching a larger interface device that allows extending beyond the corneal limbus, as described above. Steps S320, S330, S340, and S350 can each be similar to corresponding steps S220, S230, S240, and S250 described above in relation to FIG. 2. Step S360, however, can be different than previously described step S260. Step S360 can comprise creating micro-perforations through anterior sclera 460. These micro-perforations can form a type of micro-drainage channel that may allow aqueous fluid to exit anterior chamber 470 of the treatment eye 170. The fluid could flow into a subconjunctival space between the subconjuctiva 450 and sclera (anterior) 460.

In another embodiment, S360 can comprise creating micro-channel perforations, which may allow aqueous fluid to escape the anterior chamber via the uveo-scleral outflow pathway.

In another embodiment, S360 can comprise creating micro-channel perforations using laser 120 in conjunction with a subconjunctival injection/delivery system (not shown). Such a delivery system can be configured to introduce a micro-stent through the conjunctiva and into the micro-channel to the anterior chamber created by laser 120. Micro-stents of various shapes and sizes could be configured for specific use with a portion of the treatment eye. Various other devices and systems may also be required to ensure proper delivery of the micro-stents relative to the micro-channels in the treatment eye.

Other embodiments of the present disclosure will be apparent to those skilled in the art from consideration of the specification and practice of the present disclosure disclosed herein. It is intended that the specification and examples be considered as exemplary only, with a true scope and spirit of the present disclosure being indicated by the following claims.

What is claimed is:

1. A method of laser ocular surgery, comprising:
imaging a treatment eye to obtain an image of the treatment eye;
aligning a laser on a region of the treatment eye based on the image of the treatment eye; and
firing a plurality of laser pulses from the laser to ablate tissue in the region of the treatment site, wherein the tissue ablation creates micro-perforations in the region of the treatment site to incite an inflammatory reaction.

2. The method of claim 1, wherein the region of the treatment eye includes a trabecular meshwork.

3. The method of claim 1, wherein the laser includes a femtosecond laser.

4. The method of claim 1, further including coupling a patient interface device between the laser and the treatment eye.

5. The method of claim 1, further including using an imaging device to capture the image of the treatment eye.

6. The method of claim 5, wherein the imaging device includes an optical coherence tomography device.

7. The method of claim 5, wherein the imaging device includes a Schiemflug imaging device.

8. The method of claim 1, wherein the laser ablates about 180 degrees of the region of the treatment site.

9. The method of claim 1, wherein the laser ablates about 360 degrees of the region of the treatment site.

10. The method of claim 1, further including initiating remodeling of at least part of the region of the treatment site.

11. The method of claim 1, wherein the region of the treatment eye includes a portion of the anterior sclera.

12. The method of claim 11, wherein the created micro-perforations permit fluid to flow from an anterior chamber of the treatment eye to a subconjunctival space.

13. The method of claim 11, wherein the created micro-perforations permit fluid to flow from an anterior chamber of the treatment eye to an uveo-scleral outflow pathway.

14. The method of claim 11, further including introducing a micro-stent through the conjunctiva.

15. The method of claim 14, further including introducing the micro-stent through the region of the treatment site to access an anterior chamber of the treatment eye.

16. The method of claim 1, wherein aligning the laser includes optically directing a path of the laser using a reflective substrate.

17. A system configured for laser ocular surgery, comprising:
an imaging device configured to image a portion of a treatment eye;
a laser configured to generate a beam having power sufficient to create micro-perforations in the portion of the treatment eye; and
an interface device coupling the laser to the treatment eye, wherein the interface device is configured to adjust a path of the beam based on output from the imaging device.

18. The system of claim 17, wherein the imaging device is configured to output coordinate data associated with the portion of the treatment eye.
